(19) United States
(12) Patent Application Publication
(10) Pub. No.: US 2022/0344020 A1
(43) Pub. Date: Oct. 27, 2022

(54) SYSTEM AND METHOD FOR TRANSLATING MEDICAL LABORATORY DATA INTO ACTIONABLE INFORMATION

(71) Applicant: RHODES GROUP, INC., Albuquerque, NM (US)

(72) Inventors: Richard VanNess, Albuquerque, NM (US); Michael Crossey, Albuquerque, NM (US); Mark Koenig, Durham, NC (US); Patrick Prescott, West Bend, WI (US); Kathleen Swanson, Albuquerque, NM (US); Monique Dodd, Albuquerque, NM (US)

(73) Assignee: RHODES GROUP, INC., Albuquerque, NM (US)

(21) Appl. No.: 17/744,273

(22) Filed: May 13, 2022

Related U.S. Application Data
(63) Continuation of application No. PCT/US2020/060540, filed on Nov. 13, 2020.
(60) Provisional application No. 62/935,517, filed on Nov. 14, 2019.

(51) Int. Cl. G16H 20/00; G16H 10/60; G16H 10/40; G16H 50/70; G16H 50/30; G16H 15/00

(52) U.S. Cl. CPC G16H 20/00; G16H 10/60; G16H 10/40; G16H 50/70; G16H 50/30; G16H 15/00

(57) ABSTRACT

A computer implemented system and method for a) receiving in a computing system externally supplied patient data; b) comparing externally supplied patient data received by the computing system with preexisting patient data stored in a database in communication with the computing system to identify a match for a patient; c) when the patient match is identified, aggregating the externally supplied patient data with the preexisting patient data for the patient; d) incorporating current laboratory test results into the aggregated patient data for the patient; and e) analyzing the aggregated patient data with a condition algorithm specific to a diagnosis to transform the aggregated patient data into a patient actionable care plan for a user.

### CROSS-REFERENCE TO RELATED APPLICATIONS

This application is a continuation of PCT/US2020/060540, titled “SYSTEM AND METHOD FOR TRANSLATING MEDICAL LABORATORY DATA INTO ACTIONABLE INFORMATION”, filed on Nov. 13, 2020, which claims priority to and the benefit of the filing of U.S. Provisional Patent Application No. 62/935,517 titled “SYSTEM AND METHOD FOR TRANSLATING MEDICAL LABORATORY DATA INTO ACTIONABLE INFORMATION”, filed on Nov. 14, 2019, and the specification and claims thereof are incorporated herein by reference.

### BACKGROUND

Population health management requires healthcare organizations, such as providers, insurers, or health systems, to maintain a delicate balance between taking a long view of generalized patient trends and focusing personal attention on the individual and the distinctive circumstances that will influence the individual's journey towards better health.

The recommended levels or recommended ranges (ideal parameters) of biomarkers and physiological parameters (for example, weight, body mass index, respiratory rate, blood pressure, heart rate, eye movement/response for example) are established for various populations which might be based on age, sex, race etc. An individual within the assigned population is compared to the recommended levels or ranges to determine whether a patient is in “better health” as compared to any prior measure of the physiological parameter and/or biomarker for the individual and as compared to the assigned population normal for the same measurements. The comparison is also useful in making new diagnoses.

Health care providers, and physician groups are reimbursed by insurers depending on how well they meet the treatment/clinical practice guidelines and also based on the clinical outcomes for individual patient and population health.

Healthcare payers obtain higher reimbursements, improve risk management strategies, and can lower costs related to outcomes when individual and population health are reliant upon clinical practice guidelines.

Clinical practice guidelines are statements that include recommendations intended to optimize a patient's care that are informed by a systematic review of evidence and an assessment of the benefits and harms of alternative care options. Rather than dictating a one-size-fits-all approach to patient care, clinical practice guidelines offer an evaluation of the quality of the relevant scientific literature, and an assessment of the likely benefits and harms of a particular treatment. This information enables health care clinicians to select the best care for a unique patient based on his or her preferences, medical laboratory results, and a patient's history of results indicative of an increased risk of experiencing a comorbidity.

At its fundamental level, a patient's risk is a standardized evaluation process for assessing the likelihood that an individual will experience a particular outcome when the particular health, demographic and personal information of the individual is taken into consideration. To better understand patterns of what is likely to happen to individuals, insurers, physicians and physician groups need to develop insights into how each unique patient is progressing along common disease trajectories and plan interventions accordingly to ensure better outcomes.

In healthcare, these better outcomes can include decreased service utilization events, such as decreased hospital admissions and emergency department visits, or preventing the development of a certain clinical state, such as heart disease, diabetes, cancer, sepsis, kidney disease and preterm pregnancy, for example.

Assessing risk for a particular disease within a population is created by examining large cohorts of patients with similar characteristics, extracting key clinical and lifestyle indicators from those cases, and using algorithms to chart how those factors influence ultimate outcomes and based upon the risk index of the individual for a particular disease creating and providing a care plan to reflect the risk for a particular disease.

“Risk score” and “risk stratification”—the act of dividing patients into “buckets”/categories of risk based on their clinical and lifestyle characteristics—are often used interchangeably, although the two terms can have different connotations.

A risk score may indicate the likelihood of a single event, such as a hospital readmission or a poor outcome associated with a condition within the next six months, while a risk stratification framework may combine several individual risk scores to create a broader profile of a patient and his or her complex, ongoing needs.

Healthcare payers and providers can both use risk scores to estimate costs, target interventions, gauge a patient's health literacy and lifestyle choices, and try to prevent patients from developing more serious conditions or complications that could result in higher spending on their healthcare and worse health outcomes for the patient.

For providers participating in value-based care arrangements, which pair financial risk with clinical outcomes, success in identifying risk scores for individuals and ensuring good clinical outcomes can help to avoid penalties for quality of care that falls below the goal and stay on the positive side of shared savings or bonus payments. For patients, better clinical outcomes means better quality of life with lower costs for health care and decreased hospitalization for example.

Coordinating patient health information when patients often have multiple health care providers is a challenge especially when the health care providers may be in different silos such as different physician/health care provider groups, or when a patient has had different insurers over time and received care at different geographical locations (for example, different locations in a city or different cities). Often clinical laboratory results guide medical decisions and the clinical laboratory results will be associated with an individual patient at any moment in time and over a longitudinal time course. If a clinical laboratory could aggregate one or more of the following: the clinical laboratory results information for an individual patient (“A”) as the results become available for a newly requested lab test, a longitudinal history of all or select clinical laboratory results for the individual patient “A”, insurance information, recent hospitalization/discharge information for patient “A”, as well as individual patient microdata for patient “A” such as responses to fasting, ultrasound information attached to laboratory test orders, or changes in demographics, then health care providers, insurers, government entities for example or any other user entitled to the information could identify risk associated with the clinical laboratory information in near real-time (for example near real-time is an interval designated by the user if not in real-time). Real-time risk associated with clinical laboratory information for a patient is available to the user once the most recent clinical laboratory results are associated with the patient in a computing system according to an embodiment of the present invention.

### BRIEF SUMMARY

One embodiment of the present invention provides for a computer implemented method comprising receiving in a computing system externally supplied patient data. The externally supplied patient data received by the computing system is compared with preexisting patient data stored in a database in communication with the computing system to identify a match for a patient. For example, the externally supplied patient data may comprise incoming order information to a medical laboratory wherein the medical laboratory will conduct the test and report the results to the computing system, medical laboratory test results from an external user and laboratory agnostic information. When data/records/that are a match for the patient are identified, the records for the patient are aggregated for the externally supplied patient data with the preexisting patient data for the patient. Any current medical laboratory tests results are incorporated into the aggregated patient data for the patient. The aggregated patient data is analyzed with a condition algorithm to transform the aggregated patient data into a patient actionable care plan for a user. For example, the condition algorithm is selected based upon a current medical laboratory test result or a presumptive diagnosis code. The current medical laboratory test results are received by the computing system from a user. In one embodiment the medical laboratory tests are conducted by the medical laboratory controlling the computing system and the medical laboratory results are provided directly to the medical laboratory results database. Further, the aggregated patient data is matched with a user, for example, a user is a health care provider, a health care provider group, a hospital, an insurer, a government entity, a medical laboratory or a developer of the system. A user report is generated for the patient actionable care plan for each patient identified by the user. For example, a user report if provided to the user via a GUI. A patient match may be identified with traditional or probabilistic matching in one embodiment. In one embodiment, the patient actionable care plan for a condition identifies risk stratification for the patient having the condition, at risk for the condition or associated with a presumptive diagnosis code. The externally supplied laboratory agnostic information includes information about one or more patients associated with the user. For example, the user may manage a plurality of different patients such as when the user is an insurer of tens, hundreds, thousands of individuals. For a population of patients matched to the user from the computing system database, the system creates a cohort of aggregated patient data based upon the condition designated by the user.

Another embodiment of the present invention provides a computer program product for transforming medical laboratory results, the computer program product comprising a computer readable storage medium having program instructions embodied therewith, the program instructions executable by a processor to cause the processor to receive, in a computing system, externally supplied patient data. The externally supplied patient data received by the computing system is compared with preexisting patient data stored in a database in communication with the computing system to identify a match for a patient. For example, the externally supplied patient data may comprise incoming order information to a medical laboratory wherein the medical laboratory will conduct the test and report the results to the computing system, medical laboratory test results from an external user and laboratory agnostic information. When the patient match is identified, aggregate the externally supplied patient data with the preexisting patient data for the patient for which a match is identified. A current laboratory test result (that may result from an incoming order information for conducting a medical laboratory test by a medical laboratory associated with or operating or controlling the computing system) is added to and/or incorporated with the aggregated patient data for the patient. The aggregated patient data is analyzed with a condition algorithm specific to a diagnosis assigned to the patient (presumptive or confirmed) to transform the aggregated patient data into a patient actionable care plan for a user. The aggregated patient data for the patient is matched to a user. A user is selected from a patient, a health care provider, an insurer, a government entity or a developer of the system. A user report is generated for the patient actionable care plan for each patient identified by the user. For example, a user report if provided to the user via a GUI. A patient match may be identified with traditional or probabilistic matching in one embodiment. In one embodiment, the patient actionable care plan for a condition identifies risk stratification for the patient having the condition, at risk for the condition or associated with a presumptive diagnosis code. The externally supplied laboratory agnostic information includes information about one or more patients associated with the user. For example, the user may manage a plurality of different patients such as when the user is an insurer of tens, hundreds, thousands of individuals. For a population of patients matched to the user from the computing system database, the system creates a cohort of aggregated patient data based upon the condition designated by the user.

Another embodiment of the present invention provides a system comprising: at least one processor configured (appropriately programmed) to:

a) receive in a computing system externally supplied patient data;

b) compare externally supplied patient data received by the computing system with preexisting patient data stored in a database in communication with the computing system to identify a patient match for a patient;

c) when the patient match is identified, aggregate the externally supplied patient data with the preexisting patient data for the patient;

d) incorporate current laboratory test results into the aggregated patient data for the patient; and

e) analyze the aggregated patient data with a condition algorithm specific to a diagnosis to transform the aggregated patient data into a patient actionable care plan for a user. The processor is further configured to do one or more of the following: match the aggregated patient data for the patient with the user; generate a user report for the patient actionable care plan for each patient identified by the user; provide the user report to the user via a graphical user interface. The externally supplied patient data received by the computing system is compared with preexisting patient data stored in a database in communication with the computing system to identify a match for a patient. For example, the externally supplied patient data may comprise incoming order information to a medical laboratory wherein the medical laboratory will conduct the test and report the results to the computing system, medical laboratory test results from an external user and laboratory agnostic information. A user is selected from a patient, a health care provider, an insurer, a government entity or a developer of the system. A user report is generated for the patient actionable care plan for each patient identified by the user. For example, a user report if provided to the user via a GUI. A patient match may be identified with traditional or probabilistic matching in one embodiment. In one embodiment, the patient actionable care plan for a condition identifies risk stratification for the patient having the condition, at risk for the condition or associated with a presumptive diagnosis code. The externally supplied laboratory agnostic information includes information about one or more patients associated with the user. For example, the user may manage a plurality of different patients such as when the user is an insurer of tens, hundreds, thousands of individuals. For a population of patients matched to the user, the appropriately programmed processor creates a cohort of aggregated patient data based upon the condition designated by the user.

An embodiment of the present invention provides a system and method for creating aggregated patient and population health information actionable items for a user by receiving into the system current user supplied patient information such as incoming order information for a patient (“S”) (first data input) and/or current externally supplied laboratory agnostic information related to the patient “S” (second data input). The first data input and the second data input regarding patient “S” are each compared to and/or analyzed against a preexisting database of past patient data within the system to find a match. If there is a match between the current data input and the preexisting past data for patient “S” then the data is aggregated for reporting to user's having activities associated with treatment, payment and health care operations related to patient “S”. The past and current medical laboratory test results for the matched patient may be supplemented with data regarding additional forms of healthcare information such as, but not limited to, insurance eligibility files, admission-discharge-transfer data, diagnosis related grouping data, macro data supplied with laboratory test order information, and transactional claim information but not limited thereto which is user supplied in a format that is different from the first data input. The current incoming order information for patient ‘S” and/or the current laboratory results for patient “S” which are matched to preexisting past data for patient “S” if present is aggregated and the past and current patient data is analyzed with a condition algorithm that is appropriate for the diagnosis that is confirmed by the current laboratory results to produce for the user an actionable care plan for the patient with the current laboratory test results.

Embodiments of the present invention provide fora computer-implemented system and method for processing of large volumes of healthcare data to provide users improved treatment/management for the patient based upon clinical practice guidelines or other specific metrics related to timelines for recurring testing, appointments, monitoring for compliance with testing and/or appointments, management of known health conditions such as, but not limited to, renal disease, sepsis, or prenatal care, therapeutic impact on biomarkers identified in laboratory results as indicators of physiological state of patient, risk of health complications, disease progression, risk of hospitalization, to help users improve health outcomes for the patient, health care provider quality measures based upon health outcome of the patient, or operational efficiencies of health care payers amongst a population of patients or an individual patient.

Currently, a medical laboratory collects a patient's sample, processes samples, and creates large quantities of data relating to an individual's personal health information, diagnostic codes that precipitate a laboratory test or assay and data or biometrics collected as a result of the laboratory test or assay for the patient's sample that provide valuable information about an individual's health status at a point in time. The data from the point in time can be combined with the patient's prior archived health information and when combined with prior laboratory test or assay data provides valuable information about an individual's health status over a time course of minutes, hours, days, months and years (sometimes referred to herein as longitudinal history). Everything from physiological parameters, biomarkers, biologic test results, demographics, diagnosis codes, physician and patient locations, reference ranges related to the physiologic parameter, biomarker and biologic test results, and laboratory methods are stored and reported to respective customers. Customers can be one or more of the following: a patient, an individual responsible for the patient's health such as an assigned care representative, guardian, or kinship, as well as a health care provider, a state agency, an insurer, a health care system, a medical group, an epidemiologist, and a federal entity such as the Center for Disease Control and Prevention, for proper healthcare operations such as diagnosis, notification of health risk, contact tracing and health management of an individual and individuals within a population. For this reason, laboratory results are important for medical decision making [1,2] and, due to their real-time nature, [3] are at the epicenter of healthcare. Pathologists' knowledge pertaining to all aspects of how laboratory results are derived (for example the scientific methodology of sample preparation, equipment used to produce results, etc.) combined with other sources of healthcare data, such as insurance eligibility information or admission-discharge-transfer fees from hospitals, can be accepted, sorted, integrated and translated in real time with an embodiment of an automated system and method as described herein to create a real-time analysis of the health and/or personal health information of a single patient, multiple individuals within a patient population pool (sometimes referred to as population health), wherein the analysis can be customized to a single patient or patients that are common to a specific physician practice group, a single physician, an insurer, or broad population health. When multiple data points pertaining to an individual patient are properly aggregated, sorted, translated, algorithmically analyzed and presented through an intuitive graphical interface or delivered via various forms of electronic transmission, effective best-practice decisions, snapshots of patient status and actionable status can be quickly derived for populations and/or individual patients. The rapid change in an individual patient's health profile in combination with other data related to the patient impacts the status of the population health.

An embodiment of the present invention, provides a system and automated method executed thereon to provide one or more of the following: care recommendation, conveyed as “care gap” for a patient's identified health condition, health risk for an individual patient, stratify the risk for the individual patient, management recommendation for the identified risk for the individual patient, monitor the identified risk, and present population (also referred to as cohort) and individual patient-centric data to a user in light of the identified risk and care need for an individual patient or a population. Information is provided to assist any healthcare participant (e.g. a patient, healthcare provider, health system leader, quality leader, actuarial leader, researcher, insurer and/or regulatory agency personnel), in an actionable format that is easy to understand regardless of the user's medical knowledge. One aspect of the present invention provides for a means to quickly and effectively draw conclusions about population health or individual patient health and healthcare needs.

One embodiment of the present invention provides for a system for organizing medical laboratory results comprising a computing database accessible to at least one processor, one or more computer storage media for health condition analysis of an individual, and one or more data stores of medical laboratory information and a machine-readable medium storing instructions which when executed by the one or more processors, cause the system to perform one or more of the following:
from an external source, receive external information comprising external raw data associated with an individual;
store a plurality of data sets from the external information (for example external information may include, but is not limited thereto, insurance eligibility, attributed/paneled patients, ER admission, discharge, and transfer data, prescription data, claim data) separately for each individual;
combine an internal data for the individual and the external information received for the individual after specific matching and analysis of the internal data for the individual and the external information received for the individual, specified by the medical laboratory or within established care analytic purposes;
generate actionable population health information for a patient and population and;
display the actionable health information for the user in an actionable operational efficient manner for effective care management, decisional, diagnostic, prognostic, and proactive care practices.

In one embodiment, the one or more data stores include one or more of the individual's medical laboratory results, the individual's demographics, the individual's financial information, the individual's external supplied information, and data analysis techniques are disparate and may be stored in the same or different databases.

In one embodiment, the plurality of sets of data pertains to at least one or more customers of the medical laboratory wherein one customer of the one or more customers is a healthcare system, and wherein the plurality of sets of data includes at least a type of care provided to the individual through the healthcare system, and a geographic location of the individual when provided the type of care.

In another embodiment, the plurality of sets of data pertains to at least one or more customers of the medical laboratory wherein one customer of the one or more clients is a medical insurance provider, and the plurality of sets of data includes at least a type of care provided to the individual as an insured of the insurer, and geographic location of the individual when provided the type of care.

In another embodiment, the established care analytic purposes compromise medical laboratory clinical quality measures against which a healthcare organization such as the medical insurance company or health care provider is scored, measured, and evaluated.

In one embodiment, the one or more external sources of data is received in different formats, nomenclatures, or structures from a system that is external from the system for organizing medical laboratory results.

In one embodiment, the external information containing external raw data received from the external source is disparate from the medical laboratory system internal data.

For example, the external information comprising external raw data is associated with an individual and when received compromises at least clinical data sources associated with the individual, financial data sources associated with the individual, claim sources associated with the individual, admission-discharge-transfer sources associated with the individual, insurance member eligibility sources associated with the individual, patient attribution sources associated with the individual, and federal and state data sources associated with the individual.

Another embodiment provides for a population health and patient-specific analysis carried out by a computer system having at least one processor for creating patient-specific records from historical and real-time generated data, the method compromising receiving a plurality of medical laboratory generated data sources and an external raw data source associated with a population of patients; and for each data source of the plurality of data sources, using at least one processor for aggregating, analyzing, translating, each data source of the plurality of data sources into an actionable format for each patient and population of patients. For example, the receiving and translating the plurality of data sources may involve matching techniques such as probabilistic to standard field matching. Further still the processing of information by the system may include a historical data of each patient is translated into a single action point. Further still, a resultant record of patient or populations of patients is either identifiable or de-identified. In one example, de-identified data sets provide real-time data as to incidence of disease in a population with better accuracy then calling to have patient's self-identify that they identify as having the disease when asked. A better estimate of diabetes rates can assist epidemiologists, state regulators, public health entities, etc. to better understand what is happening as it relates to diseases in a community in real time.

Another embodiment provides for a computer system method for patient-centric record analysis which is carried out by having at least one processor for creating patient-specific records from historical and real-time generated data, the method compromises receiving a plurality of sets of external raw data sources associated with one or more healthcare organizations comprising attribution, eligibility, coverage, or responsibility; for each external data source, transforming each external data source into patient specific needs in accordance with treatment guidelines, medical expertise, quality regulatory compliance into one patient record; and executing at least one of a disease condition specific analysis and presented to users in an actionable risk stratified manner.

In one embodiment at least one disease specific analysis is tailored to at least one or more healthcare organizations in accordance with one or more healthcare organizational need.

In one embodiment, an analysis carried out by a computer system method wherein the computer system comprises at least one processor appropriately programmed to review a single medical laboratory result resulting from incoming order information, in combination with the patient's history of medical results stored in a database of the computer system, to determine one or more of the following: the presence, absence, progression, regression, cure, or relapse of a certain medical condition such as, but not limited to, pregnancy, chronic kidney disease, or hepatitis C, as well as an increased, decreased, absence or presence of comorbidity associated with the condition such as, but not limited to, preterm delivery, end-stage renal dialysis, or end-stage liver disease. When patient index number (PIN) is assigned to the patient, the system and method determines which user, or users, are eligible to receive output information regarding the patient. A user will supply external information/laboratory agnostic information, such as a member eligibility file or patient attribution list, to inform the system as to the patient(s) that make up the population of patients the user will receive patient data, aggregated patient data and/or population health information about. The user supplied list of patients is used to identify the user patients/members within the system database(s) and then assure only patients successfully matched to that user's supplied external information are included with information supplied to the user assuring the user's population health initiatives are limited to those patients within their user population. Further still, the system and method improves one or more of the following: response times for a user to identify current health status of aggregated patient data, identifies new insurance members prior to receiving enrollment forms, identifies current care gaps for individual patients for a condition as well as population health information for a cohort of patients sharing condition, future risk factors for the individual patient and for cohort of patients sharing condition, updates to user coding of disease based upon aggregated patient data. The information regarding aggregated patient health data and population health information for a cohort of patients sharing a condition is updated as new external information is supplied to the system. Reports regarding the updated information can be generated as the updates occur or as requested by the user for output to a GUI or output file.

Another embodiment provides for an event-related time specific analysis that is carried out by a computer system method wherein the computer system comprises at least one processor for creating patient-specific records from historical and real-time generated data, the method compromises specifying when specific outcomes occurred over a user generated timeframe. Metrics associated with change or lack thereof over a user generated timeframe is conveyed to the system. Individual and population wide needs are conveyed to the system. The information is extracted quickly.

In one embodiment, at least one disease specific trend analysis is tailored to at least one or more healthcare organizations in accordance with one or more healthcare organizational need.

Another embodiment of the present invention provides for a risk stratification analysis to be carried out by a computer system method wherein the computer system comprises at least one processor for creating patient-specific records from historical and real-time generated data, the method compromises specifying at least one disease specific optimal status for at least one, or more, patients. For the at least one or more patients, specifying at least one disease specific care need and specifying at least one disease specific potential comorbidity for at least one, or more, patients; and specifying at least one disease specific care need and potential comorbidity for at least one, or more, patients. In one example, at least one disease specific trend analysis is tailored to at least one or more healthcare organizations in accordance with one or more healthcare organizational need.

A non-transitory machine-readable medium storing instructions which, when executed by the one or more processors of a medical laboratory computing system, cause the medical laboratory computing system to perform operations comprising:
receiving a plurality of sets of external data sources associated with one or more healthcare organizations comprising attribution, paneled, eligibility, coverage, or responsibility;
for each external data source, transforming each external data source into patient specific needs in accordance with treatment guidelines, medical expertise, quality regulatory compliance into one patient record; and
executing at least one of a disease condition specific analysis and presenting to users in an actionable risk stratified manner.

One aspect of one embodiment of the present invention provides a computer system and method for transforming medical laboratory results, when the medical laboratory results (current and past) are harvested for a patient, to provide risk stratification for a patient's health condition, care guidelines to effectively treat the patient, care gaps when care guidelines missed, measure closure of care gaps, assess outcomes, user specific population health information when the patient is a member of a cohort for which a user provides treatment, payment, or health care operations.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1A-B is a block diagram of an environment for use with an embodiment of the present invention;

FIG. 2 is a flow chart of one embodiment of the present invention for the receiving external information that can be matched with laboratory generated information to assure resultant actionable information is tailored for individual users;

FIG. 3A-B illustrates risk stratification method according to one embodiment of the present invention;

FIG. 4 is exemplary report of an individual patient's aggregated health data and actionable information according to one embodiment of the present invention;

FIG. 5 is an exemplary user report of a user selected condition for a population health trend for the condition according to one embodiment of the present invention;

FIG. 6 is exemplary of how individual events attributed to a health condition are presented according to one embodiment of the present invention;

FIG. 7 is an exemplary algorithm for prenatal patient care and actional information according to one embodiment of the present invention;

FIG. 8 is an exemplary algorithm for kidney injury algorithm according to one embodiment of the present invention;

FIG. 9 is a chart illustrating longitudinal Serum Creatinine medical laboratory test results for a patient produced with one embodiment of the present invention;

FIG. 10 illustrates flow of care for a patient based upon current procedural terminology (CPT) codes used primarily to identify medical services and procedures furnished by healthcare professions (QHP), International diagnosis of disease (ICD) code which identifies a diagnosis and describes a disease or medical condition, hospitalization and diagnosis-related group code (DRG) which classifies hospital cases according to certain groups that standardizes prospective payment to hospitals and encourages cost containment initiatives in combination with longitudinal medical test results; and

FIG. 11 illustrates a method for aggregating patient data according to one embodiment of the present invention using probabilistic matching of patient data input into the system at 102 to determine if pre-existing patient data is in system database for matching.

### DETAILED DESCRIPTION OF THE EMBODIMENTS

In the following description, for the purposes of explanation, many details are set forth to deliver a thorough understanding of the present invention. However, it must be noted that the present invention may be practiced without these specific details and well-known structures and devices are shown in order to avoid unnecessarily obscuring the embodiments of the present invention.

The embodiments described herein can be used independently of one another or with any combination of the other embodiments. However, the full output and value derived from the system may not occur without embodiments working together.

As used herein “patient” means an individual who may receive or has received a procedure at a medical laboratory clinic.

As used herein “user” means a patient, physician, physician group, insurer, government entity, medical laboratory and/or personnel, or developer/engineer responsible for the maintenance of the system as described herein.

As used herein “real-time” means as fast as a laboratory test result is generated by the medical laboratory equipment and provided to the system 103.

One aspect of one embodiment of the present invention describes how clinical and anatomical laboratory data can be translated and enhanced to derive valuable and actionable insights about populations and individual patients. Embodiments of the present invention are scalable in the amount and different types of data healthcare entities derive and/or may derive in the future. Embodiments are compatible with many types of hardware and networks to ingest, store, sort, analyze, and send the various forms of information it creates. Examples of the system and method of the present invention focus on medical laboratory information; other embodiments of the system and method are agnostic in the types of healthcare data that can be ingested and are flexible to continuously provide users the information they need in a timely, easy-to-interpret format.

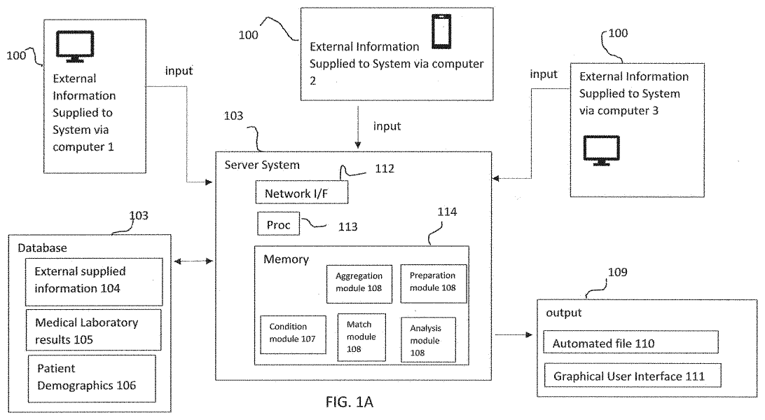

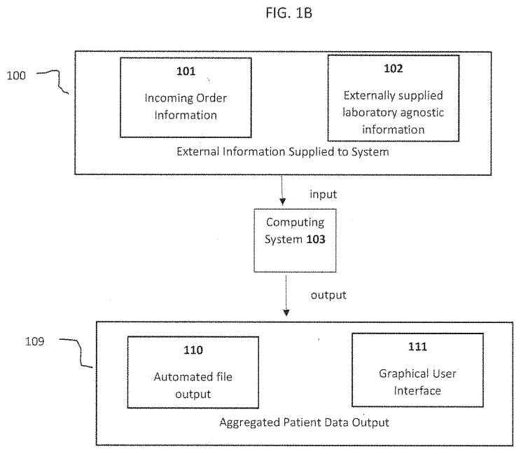

Referring now to FIG. 1A-B, an environment for one embodiment of the present invention is illustrated. User computing device 100 transmits for example external information such as incoming order information for a medical laboratory test to be performed by a medical laboratory associated with the computing system, medical laboratory test results, 101 and laboratory agnostic information 102 for input to the computing system 103, wherein the computing system comprises two or more components that communicate with one another. The user computing device interfaces to computing system 103 via a network I/F 112. Data can be stored in a database of computing system 103 and the data includes customer supplied medical laboratory data or organically derived laboratory data 105, that can be analyzed, transformed, and output 109 to customers/users such as individuals, patients, providers, care coordinators, case managers, nurses, health systems, insurers, medical groups, state (e.g. department of health) and federal regulatory entities (Centers for Medicare and Medicaid, Center for Disease Control and Prevention), epidemiologists, clinical researchers, and population health personnel. Incoming order information 101, such as electronic medical record-supplied information that may include a diagnosis code for an individual patient, and laboratory agnostic information 102 is received by the computing system 103 and is processed by one or more of the following steps: receiving external information 101 and 102 within a externally supplied information database 104, sorting the information in database 104 according to the type of information such as, but not limited to, patient demographics, fasting result, ultrasound information, ordering location. The sorted patient information in the externally supplied database 104 is paired with other data associated with the same patient within system 103 even though there is a disparity in demographic information such as the same patient has a different name but same insurance information and/or government identifying number within database 104 or another database (for example database 105 or 106) that is in communication with computing system 103. The patient information in database 104 and patient information in another database within the computing system 103 are associated with the same patient when the matching module computes that the patient information in database 104 and the patient information (medical laboratory results) in database 105 are from or belong with the same person, a unique computing system identifier is assigned to the patient. Externally supplied patient information is added to the computing system daily, for example as received as incoming order information by the computing system 103 in real time. The other data that is associated with the unique identifier for the patient may be historical data in the system 103 such as preexisting demographic data or medical laboratory test results. The matched patient information can be further processed, analyzed and stored in a database within computing system 103 such as database 104 or a different database which may be separate from other data residing in another separate database. Database 105 stores medical laboratory test results as they are received from external information 100 or generated in real time from the associated medical laboratory in computing system 103. Database 106 stores the most current patient demographic information, and data from database 104, 105, and 106 are analyzed by condition algorithm 107 via an appropriately programmed processor 113 with the resultant analyzed data stored in database 108. The action in database 108 entails aggregating most recent demographics 106 and results from the condition algorithm 107 by linking patients to their actionable information. The information is then prepared 108 into actionable information according to a user's preferences such as, but not limited, “Newly identified prenatal member” for a health insurer or “Newly pregnant patient” for a healthcare provider. The information is further analyzed 108 to determine dates of when the condition such as “New Pregnant Patient” was determined, or when a risk status was changed (e.g. “Patient Pregnancy Risk Elevated”) so it may be automatically electronically output to a user as a file 110 or displayed within a graphical user interface 111.

Incoming order information 101 can be, but not limited to, order information from electronic medical record (EMR) systems requesting medical laboratory testing for patients. The EMR system may be operated independent of the medical laboratory that receives the order for testing. The EMR system inputs information to the computing system 103 as described herein according to one embodiment of the present invention. The incoming order information 101 may include patient demographic information, assumption of health conditions in accordance with the International Statistical Classification of Diseases and Related Health Problems (ICD), diagnosis related group (DRG), visit dates, insurance information, clinic name, hospital name, and/or provider information such as a National Provider Identifier and patient location. Additional macro data may also accompany the laboratory order such as, but not limited to, the patient's fasting status, ultrasound information, patient' resistance to medication, conscious level, response level, patient's prescribed medication, and/or medication dose. These are just a few specific noted examples of data accompanying a laboratory order or incoming order information, additional data may not have been specified and not all these data fields are consistently provided.

Additional information, such as laboratory agnostic information, 102 can also be received by computing system 103. The laboratory agnostic information 102 is different from the incoming order information 101 that includes electronic medical record information. Laboratory agnostic information 102 is user specific and associates a user with an individual patient for which the user has a management relationship. For example, an insured and an insurer; a physician and a patient; a hospital and a hospital patient; a health department of a government entity such as a country or state and a citizen of the government entity such as a country or state.

One aspect of one embodiment of the present invention illustrates how a computing system 103 with historical patient data stored in a database that is in communication with the computing system 103 creates actionable information for users and through externally supplied information 102, computing system 103 aggregates user selected data specific to user's needs in real time or other time frames as specified by the user. Examples of user selected data include, but are not limited to, insurance member eligibility files, patient administration messages such as emergency room admission/discharge/transfer, attributed patient information, pharmaceutical information, financial claim data, patient problem list information, or sociodemographic information. Laboratory agnostic information 102 is supplied by users of an embodiment of a system and method of the present invention.

Incoming order information 101 and laboratory agnostic information 102 is received and sorted according to the type of data that is provided. Sorting is comprised of relational tables within the system database 103 that separate and store demographics in a specific location 106, user supplied information in another 104 and medical laboratory results in another location 105. External user information stored in database 104 is integrated with preexisting patient data (ie patient data from an earlier date in time) such as preexisting patient laboratory information stored in a database that is in communication with the computing system 103. The external information 101 and 102 received can augment/improve historical preexisting patient information stored within or in communication with the computing system 103 to improve patient data records provided to users via compliant data file 110 or a GUI interaction 111 wherein the improved patient data record includes transformed patient data files with i) aggregated patient data, ii) user specified subpopulation health information based upon aggregated patient data.

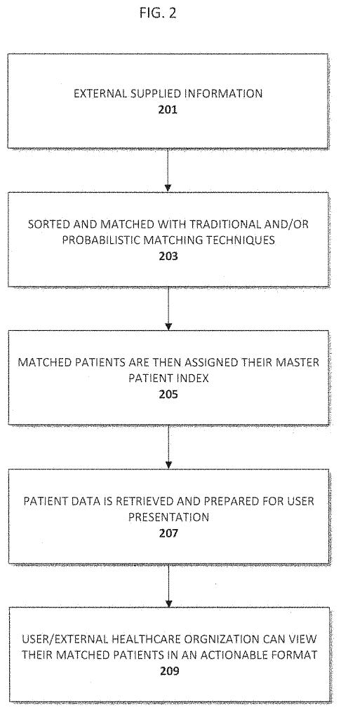

FIG. 2. illustrates one embodiment of the present invention for receiving and sorting external supplied information 201 regarding a patient “A” and matching the external supplied information received for patient “A” to a population of patient data stored in a database of preexisting patient data of the system 103 to identify preexisting patient data for patient “A”. This matching step is important in assuring the user receives information generated by system only for the user's population. As an example, the user may work for an insurance company and is interested in receiving system information about the insurance's Medicaid population. Therefore, the user would supply external information 201 such as a member eligibility file and the matching step 203, matches each of the insurance members to the patients already existing or preexisting within the computing system. When successfully matched 205, the patients receive a specific identifier attributable to the user 207 to assure the user can only receive system generated information on their population 209. This step assures protected health information is only viewed by users with the proper authority. Matching within an embodiment of the present invention can occur through traditional and/or probabilistic matching processes 203 to retrieve a historical and current context of the patient matched. A user member file is received by the computing system. The match module perform a search of database records to match patient data to the patients in the member file. Matched patients are reviewed for history of illnesses (e.g. diabetes) as well as current conditions (e.g. pregnancy) to determine care gaps and risks via a condition module. Matched patients can only be viewed by the supplying user (Health Plan A user can see Health Plan A members only. A historical context is an investigation into the patient's history of laboratory testing within a defined time period, for example life of the patient, 30 years, 20 years, 10 years, 5 years, 3 years, 2 years, 1-5 years, 5-10 years, entire historical preexisting data for a patient within a database or other time frame of medical laboratory testing results. The matching process utilizes one or more patient demographic information data such as but not limited to, first name, last name, middle name or initial, date of birth, gender, sex, social security number, patient insurance numbers (e.g. subscriber identifier), address (mailing or PO Box), phone number, and medical record number. When patient information in a preexisting patient database of the system is matched to the current external supplied information for a patient, the system assigns a master patient index identifier to the patient 205 enabling the system to retrieve, sort, evaluate, and integrate/aggregate any identified preexisting patient “A” data to generate a longitudinal historical perspective of the patient for one or more conditions. Individual patient data and/or aggregated patient data is output to a user when the user is matched to one or more patients through each master patient index. The individual patient data and/or aggregated patient data and/or population of aggregated patient data for a specified condition is matched to a user due to the patient information supplied by the user (e.g. an insurance user will be able to see the members matched from the member enrollment/eligibility file provided by the user) and a presentation is prepared for the user 207 and output to the user which may include actionable information 209. Actionable information includes, but is not limited to, instructions for what a patient with a specific condition needs currently or in the future. For example, a patient determined to be pregnant will have recommended prescriptive care based on the time of condition identification. For example, a patient determined to be pregnant and with a gestational age of 14 weeks will need a first trimester screen immediately, a gestational diabetes screen when 24 to 28 weeks into pregnancy, and a group B streptococcus infection screen at 36 weeks. Additionally, the actionable information can be tailored to the user's background and needs, for example, a patient within a provider's attributed population has just been diagnosed pregnant, therefore the actionable format would label the patient as “newly pregnant, first trimester serum screen recommended” or if the user is an insurer the actionable format would be “new prenatal, OBGYN assignment and/or prenatal care coordination recommended for meeting timeliness of prenatal care.” The risk will also be conveyed within the actionable information by informing the user the pregnant patient was also diagnosed with a urinary tract infection (UTI), a known risk for preterm delivery. In this instance, the actionable information within a provider's attributed population would label the patient as “newly pregnant, first trimester serum screen recommended, and risk of preterm delivery is high due to a UTI diagnosed within three months of conception.” This is just one example of the embodiment demonstrating how a single laboratory result indicative of pregnancy is assessed for risk, specific care needs currently and in the future, in addition to a tailored actionable format dependent on the user's background and objective.

The system and method as described herein according to one embodiment of the present invention is useful for a medical laboratory which provide laboratory results to a patient, health care provider, hospital, and other entities which provide individual patient care, management, monitoring, and verification of medical diagnoses and prognoses. The medical laboratory results 105 ordered for a patient are relied upon by a health care provider and a patient to make sound medical decisions regarding diagnoses, treatments, and management recommendations/decisions for the individual patient. The output for the aggregated patient data displays to the health care provider past care provided, current care recommended and future care needed based upon the condition algorithm. When individual patients within the system are grouped together/aggregated by common i) health care provider, ii) physician group, iii) hospital, iv) insurer, the user defined population of patients or cohort, can help decipher outbreaks, epidemics, and appropriate treatment guidelines for the medical community.

Medical laboratories may touch patients more than any other provider in the medical community enabling their patient demographic information to be more accurate and timely. This patient demographic information 106 contains, but is not limited to, patient location and contact info which has immense value in the omnipresent isolated healthcare system. With healthcare evolving more towards care management and coordination, the medical laboratory's patient demographic information 106 continues to increase in value. Patient demographic information is updated nearly every time a patient interacts with the medical laboratory enabling the medical laboratory (sometimes referred to herein as a clinical laboratory) to be the center of patient demographic knowledge.

Through peer-reviewed literature, national treatment guidelines, organization-specific clinical guidelines, and employed medical expertise, condition specific algorithms 107 are created with clinical rules that can be applied to the patient information in light of the predicted condition/proposed diagnosis code as supplied by the healthcare provided with the patient's incoming order information and/or the aggregated patient information with a different diagnosis code, condition based upon the medical laboratory results. The application of the clinical rule to the patient record determines patient compliance with regulatory or quality measures, necessary or required healthcare screenings, as well as missing care associated with certain conditions and risk of comorbidity(ies). In addition to assessing current patient status, more sophisticated condition algorithms/predictive analyses can also be created and utilized in this module 107. Predictive modeling utilizing such methods as random forest, clustering, classification, and forecasting models are just a few models that may be created within condition module 107 to further the role a medical laboratory would provide in healthcare. Such methods would position a medical laboratory, largely known as ancillary or reactive, to a tertiary or proactive placement within the healthcare delivery model. As healthcare evolves towards the value proposition, the medical laboratory needs advanced analysis to help provide more information about patients to clients and predictive analytics using sophisticated techniques such as, but not limited to, random forest, topic modeling, decision tree analysis, and deep learning neural nets. In one embodiment, algorithmic methods such as random forest, topic modeling, decision tree analysis, and deep learning neural nets create condition algorithms. In one embodiment of the present invention, a condition algorithm module 107 is separated from other modules/components of the computing system 104, 105, 106, 108. The condition algorithm can be either pre-determined through pre-established codes that are installed within the system prior to use or can be open sourced for system users to adapt within their medical laboratory environment. For example, predictive models can be biased, and an open sourced feature enables each medical laboratory to avoid such bias but adapting the models specific to the healthcare environment within which they operate. The health condition algorithm for a condition interacts with other data from the laboratory information system 104, 105, 106 to produce actionable information

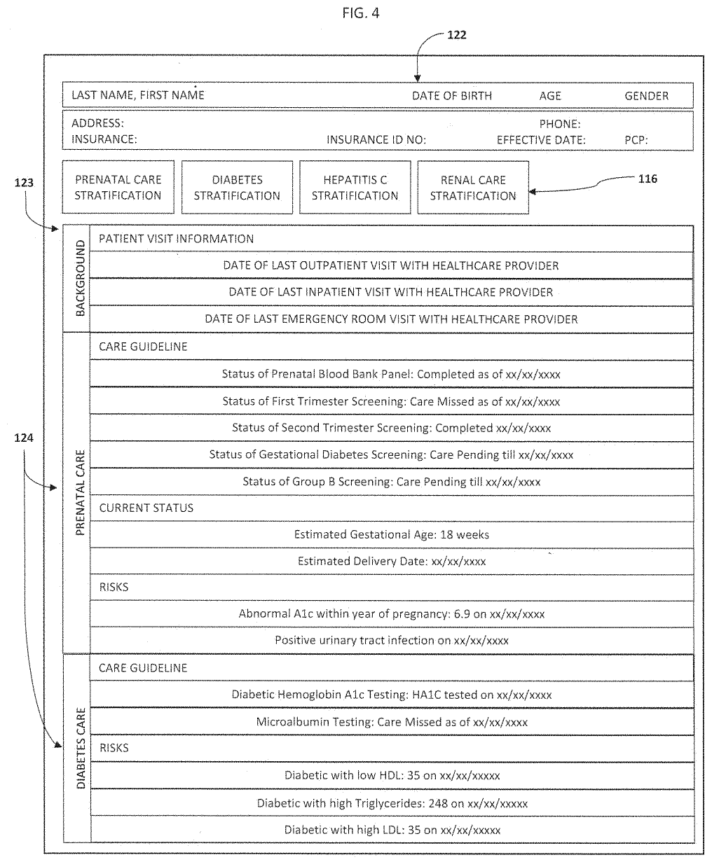

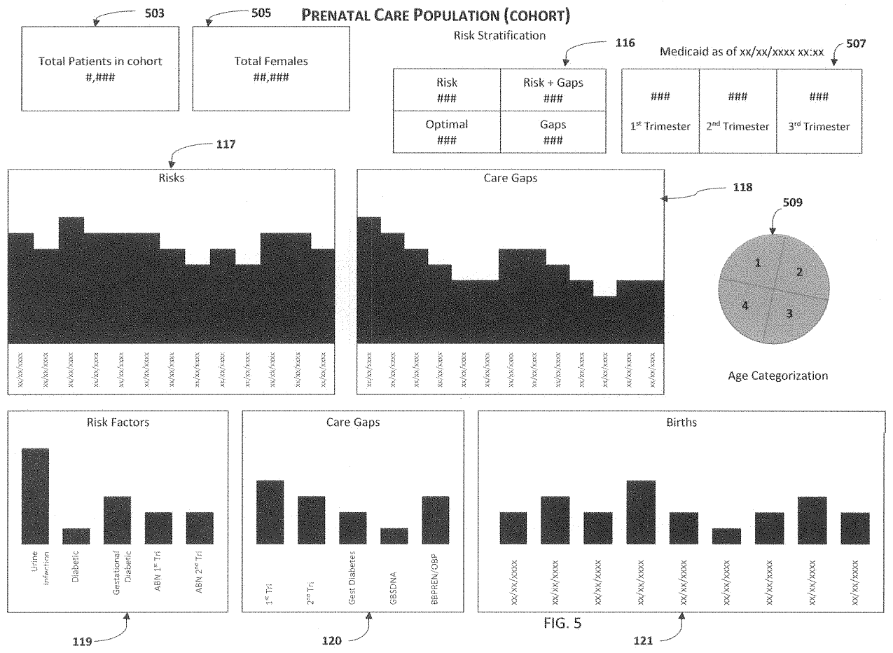

In one embodiment, the data from databases 104, 105, 106 are aggregated, combined, sorted, analyzed, and prepared for presentation through a processing module 108 as further detailed. This layer is useful in combining many forms of disparate patient information to present it as actionable information to a user. The presentation may be on individual patient information as illustrated as an example in FIG. 4 or for a population of patients as is illustrated as an example in FIG. 5. Different populations enable the medical laboratory to help guide proper medical decisions for each patient or an entire set of patients or conditions.

Resultant information (aggregated patient output data) from the data aggregation, condition analysis, and preparation module 108 is output from the system 103 and provided to the user via, for example, graphical user interface (GUI) 111 at output 109.

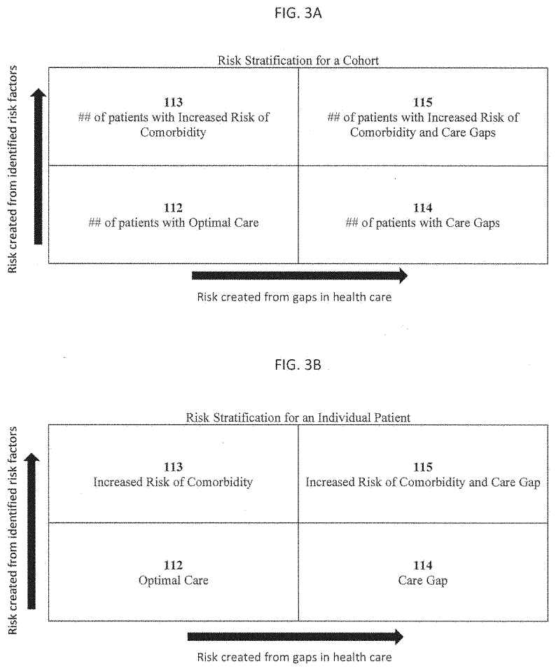

Referring now to FIG. 3A-B, one aspect of the system and method provides a risk stratification analysis FIG. 3 to help understand, at a population level, what patients are optimal in their care 112, are at risk for comorbidities 113, have care gaps 114, or have both an increased risk for comorbidities and care gaps 115. FIG. 3A illustrates risk stratification at a population level for a condition and FIG. 3B illustrates risk stratification at an individual level when a patient or a population of patients are optimal in their care 112, are at risk for comorbidities 113, have care gaps 114, or have both an increased risk for comorbidities and care gaps 115. Risk stratification for a condition is informed by one or more of the following: a condition algorithm, patient demographics, historical medical laboratory data for a patient and ongoing medical laboratory results for a patient that are aggregated.

Referring now to FIG. 4, a system and method to supply treatment templates for one or more healthcare conditions is illustrated according to one embodiment of the present invention. The aggregated patient data for a single patient is output 109 to users via a specific data file type 110 and/or through a graphical user interface 111. An individual patient-centric worklist summarizes a patient's specific needs as illustrated in FIG. 4. The actionable information enables a user to quickly decipher a patient's individual specific needs for a specific health condition(s). The patient worklist according to one embodiment of the present invention includes non-medical patient identifying information such as the latest demographic information 122 such as one or more of the following: name, date of birth, geographic location, age, gender, sex, phone number, ethnicity, national origin, economic status, employment information, insurance information derived from the patient themselves. The demographic information for a patient is stored in database 106 which enables users to reduce their disconnect with patients. This patient worklist may also incorporate the risk stratification analysis 116 (also described in FIG. 3B for an individual patient) as well the patient's most recent visit dates 123. Each table summary following the patient's background of visits 123, are specific details, needs, and overall summary of each health condition the patient is encountering 124 and for example longitudinal medical test results may be displayed to provide context for current laboratory test results. These charts are specific enough to demonstrate actual dates of previous care as well as future needs in accordance with the national treatment guidelines previously describe in the condition algorithms 107.

Referring now to FIG. 5, one embodiment of the present invention provides output 109 via a GUI 111 that illustrates the resultant aggregated patient data in a population health information format for a user defined cohort. The population health information available to a user is limited to the laboratory agnostic information 102 received in the system from a user. For example, if an insurer provided laboratory agnostic information 102 (for example a list of people in the health insurance pool of the insurer) then the patient population health information available to the insurer would be limited to the patient's insured by the insurer that are found in the preexisting patient information database in the system. The aggregated patient data can be further limited by cohort/condition. The patient's insured by the insurer would be matched to the insurer and the information provided to the insurer/user would only cover those patients insured by the insurer at the time of the report. Alternatively, the resultant data is provided in an individual patient-centric format FIG. 4. Due to life change events an insurer's insured pool may change daily thereby requiring externally supplied laboratory agnostic information provided by insurer/user and received by computing system to match this information with preexisting patient data information in the system daily.

The population health platform illustrated in FIG. 5 according to one embodiment of the present invention provides a current status of the condition, potential trends, quality compliance, and actuarial risks for a user defined population health condition/cohort for a specific condition or group of conditions. Although FIG. 5 conveys the status of a prenatal population as an example of a user defined health condition/cohort, embodiments of the present invention are not limited to such one condition. Any condition for which a medical laboratory provides a diagnosis can be displayed though an embodiment of the present invention. Examples include, but are not limited to, diabetes mellitus, hepatitis C viral infection, chronic kidney disease, cardiovascular disease, rheumatoid arthritis, oncological conditions, anemia, sepsis, and more. FIG. 5 illustrates actionable information resulting from an embodiment of the present invention data for a prenatal care population as compared to all females in the population associated with the user. Further, the information may include the risk stratification 116 for the patient population associated with a condition (ie the prenatal care population/cohort) that combines individual aggregated patient data that results from the aggregation of all laboratory data 105, 106, combined with external information 104, analyzed and aggregated patient data 108, and further analyzed with condition algorithm 107 and presented with information via output 109 to the user. Presenting the aggregate information through a risk stratification window 116 as seen in FIG. 3B for an individual patient and FIG. 3A for a patient population/cohort as defined by the user identifies the prominence of the condition and its risk status. The risk stratification graph 116 can identify percent of cohort population for a user in each category,

Still referring to FIG. 5, historical and current patient population data are also trended in an embodiment of the present invention wherein the change in risk 117 can be identified between elevated risk percent and normal risk percent on a daily basis (month/date/year or xx/xx/xxxx). Care gaps for the cohort is identified 118 on a daily basis population of cohort with care gaps and without care gaps per day. The care gaps can be identified based upon care that is complete, due, missed and pending by trimester criteria and by condition to be tested (ie gestational diabetes, group B strep for example) for 120. In addition to the actual number of risk factors 119 contributing to the potential comorbidity(ies), care gaps needing attention 120, and actual daily outcomes 121 that are occurring for a user defined population/cohort is output for a user that has provided laboratory agnostic information 102 to system 103 which user has established a right to know the information about the cohort. For example, the cohort may be the population of insured if the user is an insurance entity. For a user, the total female patients in the cohort 503 is identified all females in the user population 505. The population that is insured by government insurer 507 by date and time. A graph of age categorization for patients in cohort aged from 6-17(1), 18-24 (2), 25-34(3), 35-49(4) and age 50-75(5) 509. While the dashboard of FIG. 5 identifies the output on a daily basis, another user defined time for output can be designated since the steps for the analysis are repeatable at any interval, from seconds, minutes, hours, days or weeks, months, years. Typically, a database (preexisting patient data) of a computing system as described in an embodiment of the present invention includes hundreds, thousands, tens of thousands, millions of data in a preexisting patient database preventing a human from making the comparison, analysis, presentation in the timeframes designated.

In another embodiment the system and method provides users such as government entities, insurers, physicians and physician groups with cumulative medical laboratory data to inform each group about a population health or information about a cohort for which a user group is managing or is otherwise permitted to receive. The data associated with the population or cohort is often changing over time frames of seconds, minutes, hours and daily.

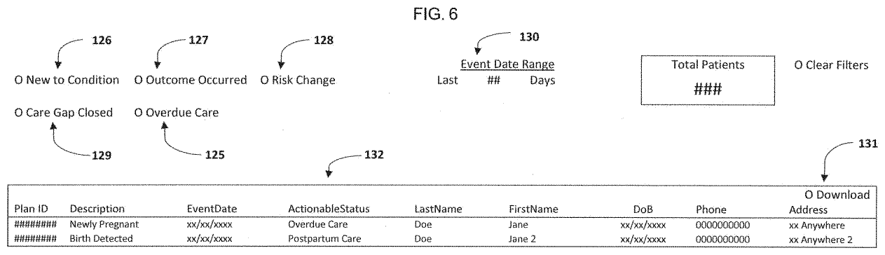

Referring now to FIG. 6, aggregated patient data is illustrated according to one embodiment of the present invention. Events are displayed over a certain period of time 130 so a user may operationally address the actionable information 132 in real-time or other time frame specified. For example, patients become noncompliant with a specific treatment or monitoring method which can be classified as overdue for care 125 which informs users that any patient classified as overdue for care need care coordination, management, or outreach to bring them back to the healthcare system for specific tests to assure their condition has not worsened. Additional events such as newly identified to the condition 126, an outcome such as a birth has occurred 127, or their risk of comorbidity has changed 128. These events help a user understand what care needs to be proactively administered to avoid noncompliance, poor outcomes, and above all patient neglect. Many users/organizations measure and incentivize providers, hospitals, clinics, health systems, and many other care providers around these events and embodiments of the system and method as described herein enables users to operationalize around actionable information provided to the user either in real time or such other user defined timeframes for improving care and outcomes. Just as important as addressing these types of events is measuring success and embodiments of the system and method help health care providers track success by identifying care gaps closed 129. All of these events can be reported through a timeline slider 130 with the ability to extract 131 at any time aggregated patient data.

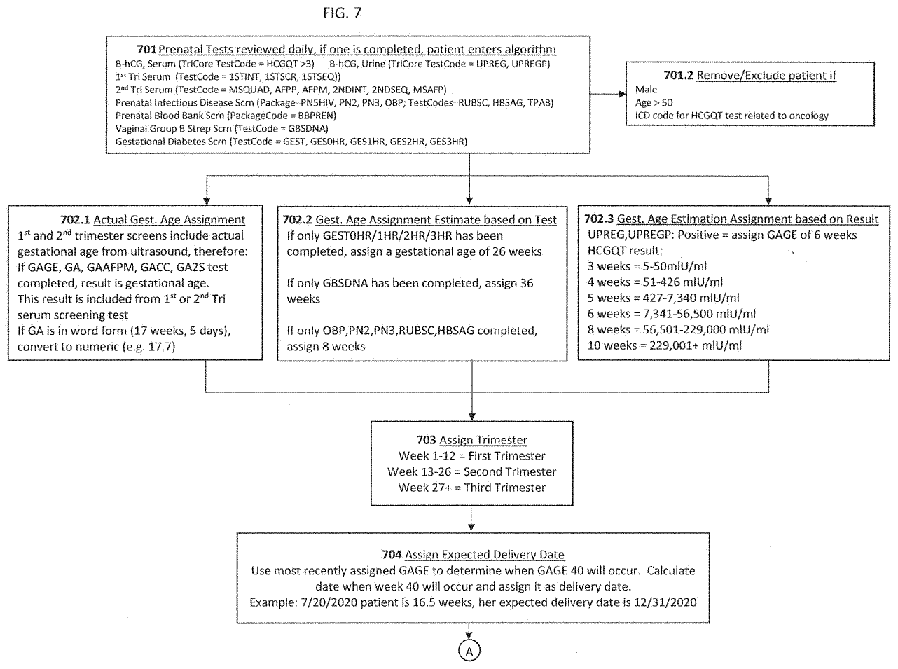

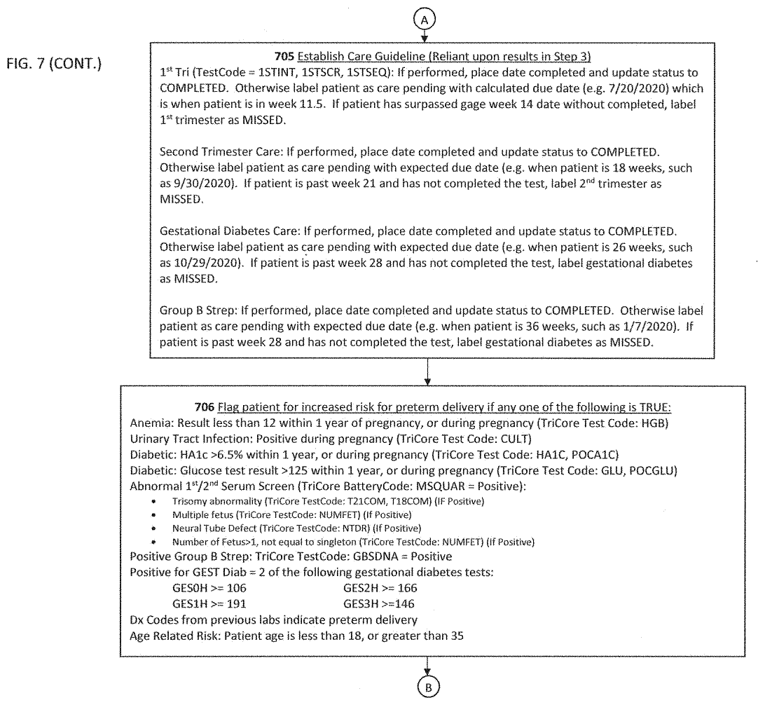

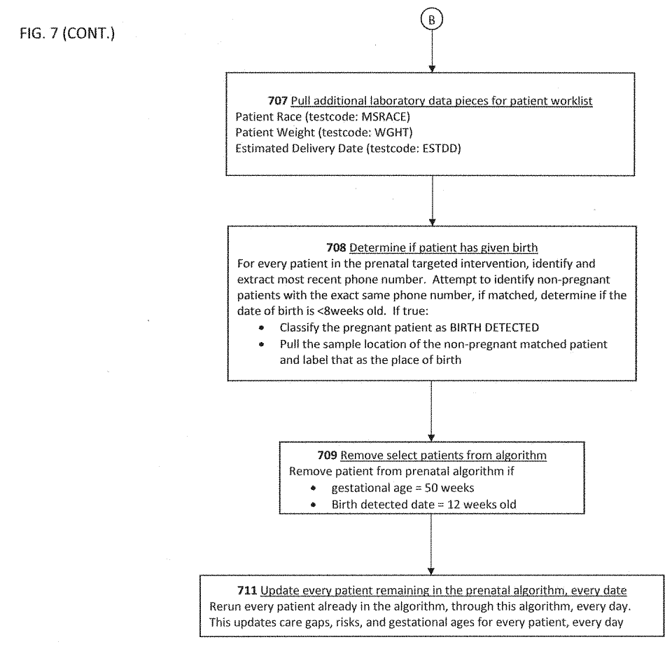

Referring now to FIG. 7, a flow chart 700 for a condition “prenatal care” algorithm is illustrated according to one embodiment of the present invention. Daily prenatal test codes received by the system 103 are reviewed 701 automatically by the system 103 and for those completed the patient information is analyzed with a prenatal care algorithm. A patient is excluded from further analysis with the prenatal care algorithm if one or more of the fields in 702 are true. For medical laboratory results identified in 703 gestational age (gestational age is also sometimes referred to in incoming orders as “GAGE”, “GA”, “GAAFPM”, “FACC”, “GA2S”) is assigned based upon the most recent test code completed for 704 or if ultrasound information 705 is not available. If ultrasound information is available 703, gestational age is assigned based upon ultrasound information. Based upon the gestational age assigned, the trimester is assigned 706. Also based upon the gestational age, the expected delivery date is assigned 707. Care guidelines are established based upon results in step 708. Increased risk for preterm delivery is identified for a patient if any of the risks 709 for a patient are identified. Additional patient information is aggregated 710 from system 103 with most recent demographic information for patient added to worklist. Every 24 hours, every patient who has been identified as pregnant is assessed for a birth 711 by having all their assigned phone numbers within 103 assessed for patients with a similar phone number and a birth date within 24 hours to determine if a new birth patient on the incoming order matches a phone number for a patient in the prenatal cohort. Since new births do not have a social security number at time of birth and some do not have a name and if the new birth patient is identified by a name the last name is sometimes provided. However, last names may not be a shared last name of the mother. Therefore, another identifier to associate the mother with the new birth patient is useful. Phone numbers associated with the prenatal care patients are reviewed to identify a match with the same phone number associated with a new birth patient. When the phone number of the new birth patient matches the phone number of a patient in the prenatal care cohort, the location where birth occurred based upon incoming order information is identified and associated with new birth patient. The new birth patient is associated with a patient in the prenatal cohort and the health plan/insurer is noticed regarding a new birth/patient to be added to the insurer's population. Actionable information for a health plan/insurer annotates the patient as “birth detected, mother needs PCP visit within 56 days” or if user is a provider, the actionable information is displayed “birth detected, mother needs postpartum assessment for depression, diabetes, and general screening.” Patients that were followed with prenatal algorithm are removed from the cohort monitored by the prenatal algorithm if the prenatal algorithm determines a gestational age of 50 weeks or if the actual date is 8 weeks or greater after the projected birth date 712. A repeat of steps 701-712 is repeated every day for a patient enrolled in the prenatal cohort. With each repeat of the analysis for the prenatal cohort, care gaps, risks and gestational age are updated daily.

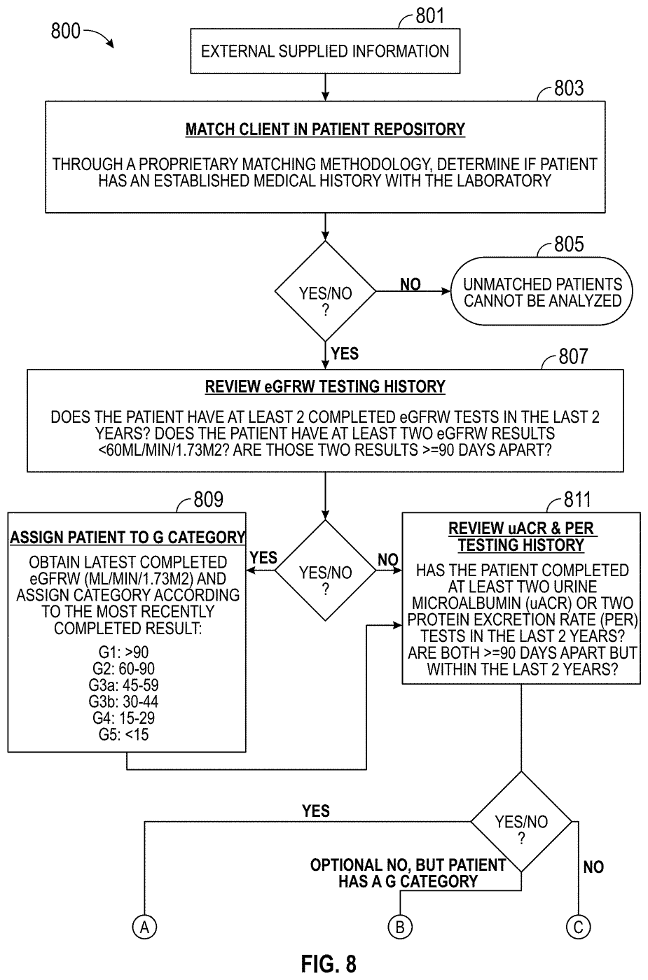

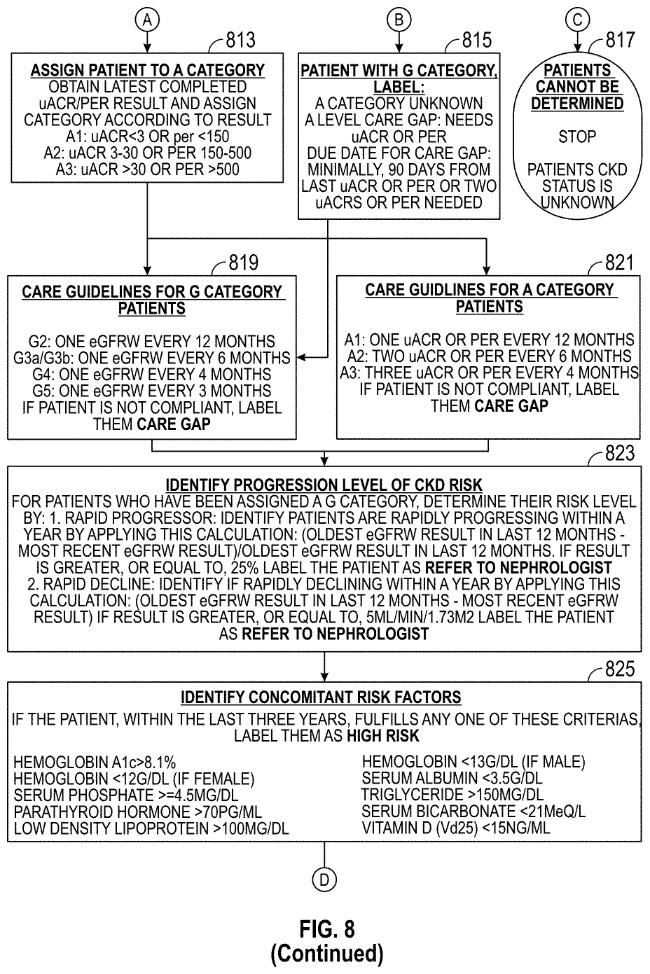

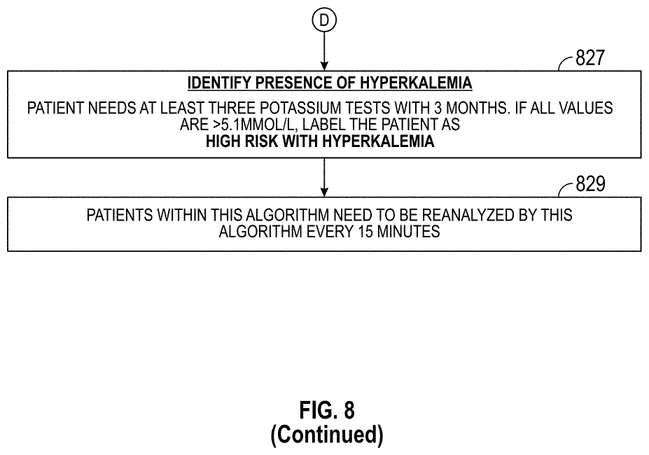

Referring now to FIG. 8, a condition algorithm specific for kidney injury 800 is illustrated according to one embodiment of the present invention. Externally supplied information related to a patient is received by system 801. For a patient referenced in the externally supplied information received, a matching algorithm compares the patient information in the system repository of patient information with the patient information in the externally supplied information to determine if the patient referenced in the externally supplied information has a history with the medical laboratory by searching for preexisting/historical patient data in the patient database 803. If there is a match between a patient referenced in the externally supplied information and the medical laboratory historical patient repository for the same patient then the historical patient information that is matched is reviewed for estimated glomerular filtration rate (eGFRW) testing history 807. If there are two estimated glomerular filtration tests and/or if the results are less than 90 days apart but within the last 2 years then assign patient to G category 809 and analyze data from G category assigned in 809 and the data in 807 without a G category assigned for uACR and PER testing history 811. If there are two urine microalbumin uACR or two protein excretion rate tests and if the tests are greater than 90 days apart but within the last two years then assign the patient to “A category” 813. If the patient does not have two urine samples as described in 811 and the patient has no “G status” assigned stop further analysis as patient's CKD status is unknown. If patient does not have two urine samples as described in 811 but has a “G category” assigned then label patient as “A category unknown” and identify care gap and/or provide actionable information 815 in patient aggregated data. Identify care guideline for G category patients 819. Identify care guideline for A category patients 821. For patient's assigned to G category, identify progression level of CKD risk 823. For patient's assigned an A category or G category identify concomitant risk factors 827, identify presence of hyperkalemia 827 and repeat the steps in 800 for every patient that qualified for analysis with this module 829 at a time interval of about evert 15 minutes.

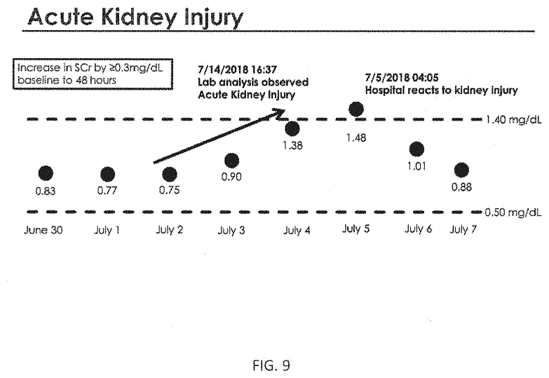

Referring now to FIG. 9, a patient care provider who examines a patient provides a presumptive diagnosis of aspiration pneumonia (ICD code J13) and orders a chest XRay and sputum sample under CPT codes 71045 and 87070 respectively to confirm the presumed diagnosis. The patient enters the hospital under the DRG code 89 for simple pneumonia and pleurisy. During the course of the hospitalization, a serum creatinine test confirms a value above an upper limit of normal indicating kidney dysfunction. However, in the absence of longitudinal historical SCr values, there is no way to know if the kidney dysfunction is acute or chronic. Further, the patient will receive a prescription Rx to treat an infection. Relying on ICD codes for monitoring a patient over time is not useful because the assignment of an ICD code is prospective and the ICD code is often inaccurate. A CPT code indicates a procedure a patient is to receive that will rule out or rule in a condition therefore it is prospective. The CPT code lacks results at the time the CPT code is assigned and therefore it is also prospective as to the condition of the patient. The DRG code is a reliable source for patient's inpatient hospital admittance and outcome but the DRG code lacks specific detail needed for hierarchical condition category (hcc). The Rx code is very specific for a patient condition but often the same medication is used to treat multiple hcc.

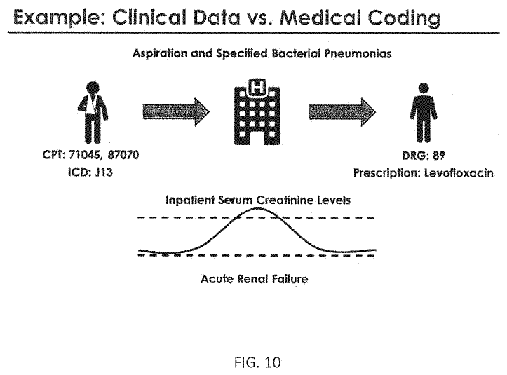

Referring now to FIG. 10, serum creatinine measurements over time is illustrated for a hospitalized patient. When the medical laboratory result reports a SCr above 1.40 mg/dl, the hospital reacts and treats the kidney injury as acute based upon the rise in SCr value. In the absence or neglect of prior values, the hospital is unable or late in the timely diagnosis and treatment of acute kidney injury as the clinical diagnosis of acute kidney injury is an increase of SCr values by >0.3 mg/dl over a 48 hour time period. Inability to react timely can result in 40% increase in an inpatient length of stay, 40% chance of developing sepsis, and 50% increase in mortality. Therefore, the health care provider of the patient in the hospital would not be aware of the acuteness and degree of increase of the SCr value in the absence of the longitudinal data that is the result of the aggregated patient data and analysis that results from the system and method as described herein. Therefore, the CPT code, the ICD code and the DRG codes may not accurately reflect the patient's actual, or newly acquired condition, and as a result of the output from the system and method according to one embodiment of the present invention, the user (ie the hospital) can appropriately bill for such a condition (as long as it is not hospital acquired), the insurer can now assign an appropriate hierarchical categorical condition for additional reimbursement from CMS, and the hospital system can more informatively follow-up with the patient to avoid the need for future dialysis.

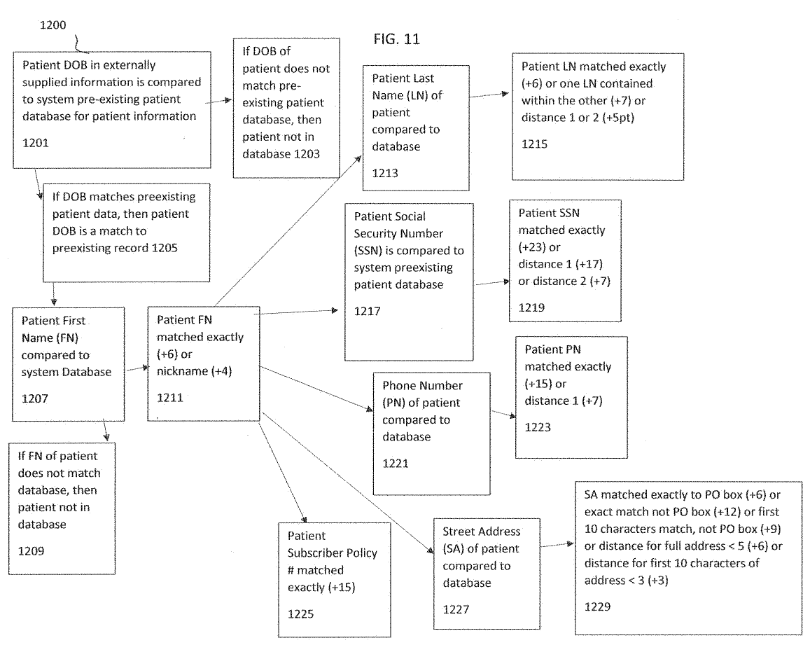

Referring now to FIG. 11, one embodiment of the patient matching method is illustrated. Probabilistic matching method 1200 is illustrated according to one embodiment of the present invention. Externally supplied information 104 received within the system is matched to pre-existing patient information in the system database that matches exactly or within acceptable mismatch to patient demographic information, name, date of birth information, insurance policy information, social security number information if a match exists. If incoming order information 101 or externally supplied laboratory agnostic information 102 for a patient includes a date of birth (DOB) for a patient, the pre-existing patient information database is queried to determine if the DOB is a match to the externally supplied information. If there is not match for the DOB for the patient in the externally supplied information database 104, then there is no match. If the DOB matches preexisting patient data in the system 1205 then patient is in preexisting patient database. The first name of the patient in the incoming order information 101 or externally supplied laboratory agnostic information 102 is compared to the preexisting patient database 1207. If the first name of the patient in incoming order information 101 or externally supplied laboratory agnostic information 102 does not match the preexisting patient database and the patient has no data to aggregate for further analysis. If there is a match of the preexisting patient data for first name with the patient identified in incoming order information 101 or externally supplied laboratory agnostic information 102 then a point system is assigned for an exact match of the first name of six points 1211. If there is match of the first name with a nickname then four points are assigned to the match 1211. Further, the last name of the patient is compared between the patient identified in incoming order information 101 or externally supplied laboratory agnostic information 102 and the preexisting patient database 1213. If the patient last name that is being compared between the information in the externally supplied information database 104 and the preexisting patient database is an exact match then six points is assigned 1215. If there is more than one last name for the patient and the last name being compared is contained with the other last name then seven points are assigned and if there is a mismatch of only 1 or 2 characters that may occur due to typographical or phonetic spelling then five points are assigned 1215. Further, the social security number (SSN) or other government assigned number of the patient is compared between the patient identified in incoming order information 101 or externally supplied laboratory agnostic information 102 with the data in the preexisting patient database 1217. If the patient SSN that is being compared between the information in the externally supplied information database 104 and the preexisting patient database is an exact match then twenty three points is assigned 1219 but if there is a mismatch of 1 character then seventeen points are assigned and if there is a mismatch of 2 characters then seven points are assigned. Further, the phone number (PN) of the patient is compared between the patient identified in incoming order information 101 or externally supplied laboratory agnostic information 102 and the preexisting patient database 1221. If the patient PN that is being compared between the information in the externally supplied information database 104 and the preexisting patient database is an exact match then fifteen points are assigned 1223 but if there is a mismatch of 1 character then seven points are assigned. Further, the address either PO box or residence number (SA) of the patient is compared between the patient identified in incoming order information 101 or externally supplied laboratory agnostic information 102 and the preexisting patient database 1227. If the patient PO box that is being compared between the information in the externally supplied information database 104 and the preexisting patient database is an exact match then six points are assigned or exact match for street address then twelve points are assigned 1229. But if first 10 characters match, not PO box nine points assigned or if mismatch for full address <5 six points are assigned and if distance for first 10 characters of address <3 then three points are assigned 1229. If the total points assigned for the probabilistic match of the patient information in the fields that are matched to the fields for a patient in the preexisting patient database is at least 24 points or between 20-24 points or between 15-20 points then the patient in the preexisting patient database is a match with the patient identified in the externally supplied information database and the data for the patient in the preexisting patient database and the patient in the incoming order information 101 or externally supplied laboratory agnostic information 102 may receive a master patient index and the data 104 and other data for the patient may be aggregated.

Referring now to FIG. 1, it will be appreciated that the system described and illustrated in FIG. 1 represents one embodiment of the present invention embodiments. For example, present invention embodiments may include any number of computer or other processing systems (e.g., client or user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer/computing system or other processing systems employed by the present invention embodiments may be implemented by any number of any personal or other type of computer or processing system (e.g., desktop, laptop, tablet, PDA, mobile devices, etc.), and may include any commercially available operating system and any combination of commercially available and custom software (e.g., browser software, communications software, server software, profile generation module, profile comparison module, etc.).

It is to be understood that the software (e.g., condition algorithm 104, a matching, aggregation, analysis algorithm/module 108 of the present invention embodiments may be implemented in any desired computer language which language could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The algorithm/module/software of the present invention embodiments (e.g., condition algorithm/module 107, mapping, aggregation, analysis, and preparation module 108, may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the server and/or client systems. The database systems and/or storage structures may be remote from or local to the computer or other processing systems, and may store any desired data (e.g. medical laboratory test results, demographic data, incoming order information etc).

The present invention embodiments may employ any number of any type of user interface (e.g., Graphical User Interface (GUI), command-line, prompt, etc.) for obtaining or providing information, where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.’

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Example 1

A non-randomized feasibility study was conducted for the purpose of evaluating the viability of using a medical laboratory's patient data for longitudinal laboratory insights to augment current care coordination services. Data was collected for three primary objectives involving the identification and delivery of prenatal and post-partum care. Primary objectives associated with process outcomes included:
1. Identification of members in the first trimester of pregnancy to support early initiation of prenatal care.
2. Identification of births within 24 hours of parturition as a mechanism to facilitate postpartum care.
3. Identification of members lacking ongoing prenatal care associated with laboratory testing as recommended by treatment guidelines.
   Secondary objectives associated with process and clinical outcomes included:
1. Identification of members diagnosed with pregnancy during an emergency room visits in order to identify those who require obstetric care.
2. Preterm births defined as births occurring prior to 37 weeks of gestation.
3. Neonatal intensive care unit (NICU) admissions and length of stay.
   The intervention was the use of a prenatal targeted intervention module (TIM) analysis to supplement, but not replace, existing insurance care management processes. Prior to the use of the TIM analysis, insurance care coordinators would identify patients through claims and prescription data. Upon potential identification of a pregnancy, insurer would make at least 3 outreach attempts, each a week apart, to initiate contact with the patient for enrollment in their prenatal care processes. Members successfully enrolled in a care management program entitled “Special Beginnings,” were followed throughout pregnancy and the post-partum period. High-risk patients were referred for care coordination services and followed more closely depending on the conditions being monitored. The prenatal TIM, powered by algorithms analyzing clinical laboratory test results, was used as the primary source of information for identifying pregnancies, high-risk patients, and gaps in care (details below) and matched with external source information provided by the insurer, namely patient's within the insurer's population of coverage. A feature of the TIM is the inclusion of comprehensive, longitudinal laboratory test results, associated metadata, and treatment locations in addition to a method to identify births within 24 hours of parturition. These clinical insights were provided weekly as a real-time decision support tool in a longitudinal, patient-centric format. The TIM did not replace insurer's current care management processes, did not contain information found in claims data, and was not integrated into other insurer analytics programs.

The prenatal TIM utilized guidelines published by the American Academy of Pediatrics and ACOG for prenatal care. Actionable insights provided by the prenatal TIM included identification of pregnancy, actual or estimated gestational age, selected prenatal-associated laboratory tests completed or not completed, estimated date of delivery, and the occurrence of a birth. The prenatal TIM also stratified pregnant women for risk factors due to concomitant health conditions (e.g. diabetes) or medical history (e.g. advanced maternal age) and/or gaps in care, defined as missing or incomplete prenatal laboratory tests appropriate for gestational age. The prenatal TIM identified the trimester of the pregnancy based on the completion and timing of specific prenatal laboratory tests. For example, screening for gestational diabetes mellitus is recommended between week 24 and 28 of pregnancy, thus a patient who completed this test was determined to be in the second trimester. Births and NICU occupancy were identified using information on the location of phlebotomy services provided to the infant immediately after birth. For infants admitted to the NICU, the length of stay was determined by calculating the time difference between the date of the first and last phlebotomies performed in the NICU.

Patients for the feasibility study were identified by matching a monthly insurer member enrollment file to medical laboratory's data repository. Required identical data elements for a match included first name, last name, sex, date of birth, and social security number. All pregnant insurer's Medicaid members identified through one or more laboratory pregnancy tests were included in the study sample. Successful matches in the medical laboratory's data repository were used to populate the prenatal TIM.

Prenatal care coordinators attempted to contact all insurer's members identified by the prenatal TIM for enrollment in prenatal care management and coordination services. To support the care coordinators' efforts, the most current demographic information for each member available to medical laboratory was also supplied within the TIM. The demographic information assisted insurer to establish a continuum of care during the pregnancy in accordance with the Code of Federal Regulation (CFR) Title 45 Section 164.506 entitled “Uses and disclosures to carry out treatment, payment, or health care operations”. CFR 45 section 164 also defines a user that may receive aggregated patient data/information as described herein.

The primary analysis examined the ability of the TIM analysis to meet primary objectives compared to the Healthcare Effectiveness Data and Information Set (HEDIS) metrics developed and maintained by the National Committee for Quality Assurance or other comparator data before implementation. An analysis for the secondary objectives was conducted to examine differences in clinical outcomes for two groups of insurer 1 members identified by the TIM analysis. Both groups of members were identified using medical laboratory prenatal TIM and received usual insurer member outreach processes for care coordination services. Group A members had evidence of ongoing prenatal care (defined as the completion of one or more prenatal-associated laboratory tests following notification of insurer for care coordination). Group B members had no evidence of ongoing prenatal care. Since lack of prenatal care could result in adverse clinical outcomes, establishing a true control group by excluding insurer members was not considered.

Over the eleven-month study, the prenatal TIM identified 1,355 pregnant insured Medicaid members in real-time. Nearly two thirds of the women lived in metropolitan areas, 28% had at least one prenatal risk factor defined by treatment guidelines, and the mean maternal age was 28.1 years. The prenatal TIM identified 77% of all pregnancies in the first trimester compared to 63% previous reported as a HEDIS metric for Medicaid members the previous year. Since women may not seek prenatal care in the first trimester, the study sought to identify pregnancies at any time up to the delivery. 12% of pregnancies were identified in the second trimester and 11% in the third trimester. Not all pregnancies were successfully linked to a birth. A total of 488 births (36%) were detected within 24 hours. The mean gestational age at delivery was 38.5 weeks (range, 30.4-41.3 weeks).

Emergency room visits were more likely to occur among Group B patients who utilized the service at least once significantly more often than Group A patients (p=0.03) (Table 3). Of the 488 births identified, reliable gestational ages at delivery from laboratory results were available for 159 infants. Using known gestational age, Group A had a lower rate of preterm delivery (11.4%) compared to Group B (19.7%). The location of an infant's first phlebotomy was used to determine NICU occupancy. Using this criterion, the location of 435 of the 488 delivered infants was identified (Group A, N=177; Group B, N=258). The rate of NICU admissions for Group A (10.7%) was significantly less than that of Group B (18.2%) (p=0.03). The mean length of stay in the NICU for Group A was 16.6 days which was not significantly different from the 12.3 days observed for Group B (p=0.39). When a single outlier in Group A the study group was removed due to a prolonged stay of 94 days, the mean lengths of stays were identical at 12.3 days.

Clinical laboratory data has the advantage of providing real-time and longitudinal insights that can drive medical decisions to impact care. While many laboratory-based initiatives focus on a single intervention point using one laboratory result, measuring the value of the lab across the entire spectrum of a disease or condition (including screening, diagnosis, management, monitoring, and clinical response post-intervention) can be an effective tool to support care management. Embodiments of the present invention seek to provide meaningful clinical diagnostic insights for population health initiatives that result in improved short- and long-term patient outcomes and drive cost-effective care.

Insurer reported that 65% of the pregnancies identified through the laboratory insights were not found in claims and that the number of members enrolled in their pregnancy education program increased 35%. Through the use of these laboratory insights, insurer was able to identify more pregnant members, increase the number of women receiving early prenatal care, monitoring of that care was ongoing during the pregnancy, and impacted the likelihood of an uncomplicated gestation. With 77% of all pregnancies detected in the first trimester, insurer improved their HEDIS prenatal care measure for women having a health care visit in the first trimester of pregnancy.

Note that in the specification and claims, “about” or “approximately” means within twenty percent (20%) of the numerical amount cited. All computer software disclosed herein may be embodied on any computer-readable medium (including combinations of mediums), including without limitation CD-ROMs, DVD-ROMs, hard drives (local or network storage device), USB keys, other removable drives, ROM, and firmware. In at least one embodiment, and as readily understood by one of ordinary skill in the art, the apparatus according to the invention will include a general or specific purpose computer or distributed system programmed with computer software implementing the steps described above, which computer software may be in any appropriate computer language, including C++, FORTRAN, BASIC, Java, assembly language, microcode, distributed programming languages, etc. The apparatus may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements.

Computer-readable media include both volatile and nonvolatile media, removable and nonremovable media, and contemplates media readable by a database, a switch, and various other network devices. By way of example, and not limitation, computer-readable media comprise media implemented in any method or technology for storing information. Examples of stored information include computer-useable instructions, data structures, program modules, and other data representations. Media examples include, but are not limited to information-delivery media, RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These technologies can store data momentarily, temporarily, or permanently.

Database: A broad term for any data structure for storing and/or organizing data, including, but not limited to, relational databases (Oracle database, mySQL database, etc.), spreadsheets, XML files, and text file, among others.

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

Computing device(s) are generally controlled and coordinated by operating system software, such as iOS, Android, Chrome OS, Windows XP, Windows Vista, Windows 7, Windows 8, Windows Server, Windows CE, Unix, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

One or more embodiments of the present invention provide a computerized system, methods, and computer-readable media for use in facilitating clinical decision making, and in particular, knowledge integration for facilitating patient care, improving provider quality, decreasing patient care gaps, identifying risk for comorbidity(ies), and stratifying patient populations for user defined conditions. For example, when the user is an insured and the patient population is a portion of the pool of insured such as only applying this embodiment to the Medicaid population and not Medicare.

Although the invention has been described in detail with particular reference to these embodiments, other embodiments can achieve the same results. Variations and modifications of the present invention will be obvious to those skilled in the art and it is intended to cover in the appended claims all such modifications and equivalents. It should be noted that embodiments of the present invention are not limited to the manipulation of particular data content, but in fact improve the functioning of the computer system regardless of what the data describes. The entire disclosures of all references, applications, patents, and publications cited above are hereby incorporated by reference.

### REFERENCES

1. Forsman, R. W. Why is the Laboratory an Afterthought for Managed Care Organizations? (1996) Clin Chem. 42: 813-816
2. Laposata M E, Laposata M, Van Cott E M, Buchner D S, Kashalo M S, and Dighe A S. Physician Survey of Laboratory Medicine Interpretive Service and Evaluation of Interpretations on Laboratory Test Ordering. (2004) Arch Pathol Lab Med. 128(12): 1424-1427
3. Ho Ahn C, Yoon J W, Hahn S, Mon M K, Park K S, Cho Y M. Evaluation of Non-Laboratory and Laboratory Prediction Models for Current and Future Diabetes Mellitus: A Cross-Sectional and Retrospective Cohort Study. (2016) PLoS One. 11(5): e0156155

1. A computer implemented method for improving retrieval of data stored in a computer memory comprising:
a) receiving in a computing system externally supplied patient data;
b) comparing the externally supplied patient data received by the computing system with preexisting patient data stored in a database in communication with the computing system to identify a match for a patient;
c) for the patient match identified, aggregating the externally supplied patient data with the preexisting patient data for the patient;
d) incorporating current laboratory test results into the aggregated patient data for the patient; and
e) analyzing the aggregated patient data with a condition algorithm specific to a diagnosis to transform the aggregated patient data into a patient actionable care plan for a user.

2. The method of claim 1, further comprising:
matching the aggregated patient data for the patient with the user.

3. The method of claim 1, further comprising:
generating a user report for the patient actionable care plan for each patient identified by the user.

4. The method of claim 3, further comprising:
providing the user report to the user via a graphical user interface.

5. The method of claim 1, wherein the patient match is identified with traditional or probabilistic matching.

6. The method of claim 1, wherein the externally supplied patient data comprises incoming order information and laboratory agnostic information.

7. The method of claim 1, wherein a user is selected from a patient, a health care provider, an insurer, a government entity or a developer of the system.

8. The method of claim 1, wherein the patient actionable care plan for a condition identifies risk stratification for the patient.

9. The method of claim 1, wherein the externally supplied laboratory agnostic information includes information about one or more patients associated with the user.

10. The method of claim 2, further comprising:
from a population of patients matched to the user, creating a cohort of aggregated patient data based upon the condition designated by the user.

11. A computer program product for transforming medical laboratory results, the computer program product comprising a computer readable storage medium having program instructions embodied therewith, the program instructions executable by a processor to cause the processor to:
a) receive in a computing system externally supplied patient data;
b) compare externally supplied patient data received by the computing system with preexisting patient data stored in a database in communication with the computing system to identify a patient match for a patient;
c) when the patient match is identified, aggregate the externally supplied patient data with the preexisting patient data for the patient;
d) incorporate a current laboratory test result into the aggregated patient data for the patient; and
e) analyze the aggregated patient data with a condition algorithm specific to a diagnosis to transform the aggregated patient data into a patient actionable care plan for a user.

12. The computer program product of claim 11, further comprising:
match the aggregated patient data for the patient with the user.

13. The computer program product of claim 11, further comprising:
generate a user report for the patient actionable care plan for each patient identified by the user.

14. The computer program product of claim 13, further comprising:
provide the user report to the user via a graphical user interface.

15. The computer program product of claim 11, wherein the patient match is identified with traditional or probabilistic matching.

16. The computer program product of claim 11, wherein the externally supplied patient data comprises incoming order information and laboratory agnostic information.

17. The computer program product of claim 11, wherein a user is selected from a patient, a health care provider, an insurer, a government entity or a developer of the system.

18. The computer program product of claim 11, wherein the patient actionable care plan identifies risk stratification for the patient.

19. The computer program product of claim 12, wherein the externally supplied laboratory agnostic information includes information about one or more patients supplied by the user.

20. The computer program product of claim 12, further comprising:
from a population of each patient matched to a user, create a cohort of aggregated patient data based upon a user designated condition.

21. A system comprising: at least one processor configured to:
a) receive in a computing system externally supplied patient data;
b) compare externally supplied patient data received by the computing system with preexisting patient data stored in a database in communication with the computing system to identify a patient match for a patient;
c) when the patient match is identified, aggregate the externally supplied patient data with the preexisting patient data for the patient;
d) incorporate current laboratory test results into the aggregated patient data for the patient; and
e) analyze the aggregated patient data with a condition algorithm specific to a diagnosis to transform the aggregated patient data into a patient actionable care plan for a user.

22. The system of claim 21, wherein the processor is further configured to:
match the aggregated patient data for the patient with the user.

23. The system of claim 21, wherein the processor is further configured to:
generate a user report for the patient actionable care plan for each patient identified by the user.

24. The system of claim 23, wherein the processor is further configured to:
provide the user report to the user via a graphical user interface.

25. The system of claim 21, wherein the patient match is identified with traditional or probabilistic matching.

26. The system of claim 21, wherein the externally supplied patient data comprises incoming order information and laboratory agnostic information.

27. The system of claim 21, wherein a user is selected from a patient, a health care provider, an insurer, a government entity or a developer of the system.

28. The system of claim 21, wherein the patient actionable care plan identifies risk stratification for the patient.

29. The system of claim 21, wherein the externally supplied laboratory agnostic information includes information about one or more patients supplied by the user.

30. The system of claim 21, wherein the processor is further configured to:
from a population of each patient matched to a user, create a cohort of aggregated patient data based upon a condition designated by the user.
